(19) United States
(12) Patent Application Publication
(10) Pub. No.: US 2024/0252742 A1
(43) Pub. Date: Aug. 1, 2024

(54) WEARABLE DRUG RELEASING PUMP AND METHOD FOR RELEASING DRUG THERE THROUGH

(71) Applicant: Sunil SINGH, Haryana (IN)

(72) Inventor: Sunil SINGH, Haryana (IN)

(21) Appl. No.: 18/561,299

(22) PCT Filed: May 16, 2022
(86) PCT No.: PCT/IN2022/050471

§ 371 Date: Nov. 16, 2023

(51) Int. Cl. A61M 5/142

(52) U.S. Cl. CPC A61M 5/14248; A61M 2005/14252; A61M 2205/0266

(57) ABSTRACT

The present disclosure relates to a wearable drug-releasing pump (1000) and a method (2000) thereof for releasing a drug in a user's body therethrough. The wearable drug-releasing pump (1000) is configured to provide drug dosages at predefined intervals of time and at a predetermined rate such that the user gets access to consistent, around-the-clock uninterrupted drug delivery. Further, the present disclosure relates to a method (2000) for providing of a drug into a patient using the wearable drug-releasing pump.

### FIELD OF INVENTION

The present disclosure generally relates to the field of medical devices, more specifically the present disclosure relates to a wearable drug-releasing pump and a method for releasing drug such as insulin into user's body therethrough.

### BACKGROUND OF THE INVENTION

In general, insulin is injected subcutaneously into the diabetes patient twice or thrice a day. The dosage of insulin required may vary from a patient to patient depending upon physiological conditions thereof. In addition, the dosage required may vary at different intervals of consumption thereof. For example, more insulin needs to be injected after meals and similarly, the insulin dosage may be different for fasting, before breakfast, after breakfast, before lunch, after lunch, before dinner, and after dinner.

Additionally, insulin via pumps is often delivered through tubing that can kink, catch, and/or detach. These tubing issues, along with the current size of available pumps, often compromise discreet and convenient use.

The patients with controlled diabetes can consume insulin in the form of tablets. However, the patients who suffer from Type 1 Diabetes may require insulin provided in fats thereof, dosage and duration thereof varies from patient to patient. Such insulin dosages may be provided through injections. Such injections may have needles which can be use and throw. As such needles may be costly, patients prefer using reusable needles. However, in both the cases, the patient needs to prick skin multiple times a day especially when they require more than one dose. Such repetitive pricking causes pain, trauma, skin redness, and sometimes infections also, and so on. In addition, the needle of the injection has to be in proper orientation and also requires proper pressure to apply thereon to provide proper dosage of insulin into the fat of the patient. There may be chances that insulin can bulge out if improper pressure or angle between the insulin injection and the skin is inaccurate. Hence, the patient either needs to be technically expert of injecting insulin by himself or should take under medical supervision. In some instances, it may be difficult for the patients to take insulin when outdoors. Insulin has to be stored in proper conditions which actually may be difficult for the patients when outdoors. Sometimes, outdoor environmental conditions may also not be apt for providing insulin injections.

There have been a number of conventional arts developed which pertain to tubeless wearable drug releasing pumps such as insulin releasing patches. Such patch pumps involve no tubing, and are small, lightweight, completely or partially disposable, capability of being worn and manipulated discreetly under clothing. However, sizes of such patch have always been a concern as the patient needs to wear it continuously for 3 days at least. These may be inconvenient for the patient to wear such large sized patches for so many days. There have been challenges to accommodate all the elements involved in larger patches in a small patch. Further, the existing arts may have higher economic cost as the patient may require using such patches for their entire long life. Further, existing patches are not smart enough to provide dosages in case of emergencies.

Therefore, there exists a need for developing devices that are smart, small, low cost and environment friendly for providing drug to the user's.

### SUMMARY OF THE INVENTION

In view of the foregoing, a wearable drug releasing pump and a method for releasing drug therethrough. The wearable drug releasing pump includes an encoder plate, one or more SMA wires operatively coupled to the encoder plate and adapted to exhibit a variation in length such that upon variation of the length of one or more SMA wires facilitates the encoder plate to sway further a syringe unit includes a syringe, a gear coupled to the encoder plate, and is adapted to rotate, upon sway of the encoder plate, a lead screw having a first end that is coupled to the gear, and configured to rotate, upon rotation of the gear; and a plunger coupled to the lead screw, and configured to translate upon rotation of the lead screw within the syringe such that the plunger, upon translation is adapted to dispense a pre-determined quantity of a drug from the syringe. The wearable drug releasing pump also includes a plurality of studs that are configured to fix a one end of the one or more SMA wires; and a one or more pulleys that are placed adjacent to the encoder plate, that are configured to provide a tension to the one or more SMA wires such that another end of the one or more SMA wires is fixed to the studs projecting from the encoder plate. The wearable drug releasing pump also includes the encoder plate which further includes a one or more pawls projecting from the encoder plate such that the one or more pawls are configured to rotate the gear, upon sway of the encoder plate. The wearable drug releasing pump also includes the syringe unit which further a lead screw having a second end that is on a opposite side of the first end of the lead screw extends within the syringe. The wearable drug releasing pump also includes the syringe unit which further includes a fill port; that is enables to fill the wearable drug releasing pump by means of injection. The wearable drug releasing pump also includes an electronic unit that is placed adjacent to the encoder plate, the electronic unit is configured to provide an electric current to heat the one or more SMA wires, upon heating the one or more SMA wires is adapted to exhibit a variation in length such that upon variation of the length of one or more SMA wires facilitates the encoder plate to sway. The wearable drug releasing pump also includes a needle and canula unit such that the needle and canula unit further includes a needle holder adapted to hold a needle, a cannula holder adapted to hold a cannula, and a spring that is disposed between the needle holder and the canula holder to retract back the needle. The wearable drug releasing pump also includes a canula insertion unit such that the cannula insertion unit further includes a third locking stud, a fourth locking stud, a third SMA wire that is coupled to the third locking stud at one end and the fourth locking stud on the other end; a needle insertor, a torsion spring coupled to the needle insertor; wherein, variation of length of the third SMA wire releases the third locking stud that activates the torsion spring by which the needle inserter pushes down the needle holder which further pushes down the needle and the cannula into a subcutaneous tissue of a user body. The wearable drug releasing pump also includes a Bluetooth Low Energy (BLE) enabled application to connect a user with the wearable drug releasing pump.

In some aspects, a method for providing of a drug into a user's body by a wearable drug-releasing pump, the method includes steps; a). introducing drug into the wearable drug releasing pump using an injection via a fill port; b). applying of the wearable drug releasing pump onto a user's body; c). varying of a length of one or more SMA wires; d). swaying of an encoder plate by varying in length of the one or more SMA wire; e). rotating of a gear by swaying of the encoder plate; f). translating of a plunger by rotating of the gear; g). dispensing of a pre-determined quantity of a drug from a syringe by translating of the plunger; h). detaching of a third locking stud of a needle insertor; i). inserting of a needle and cannula further into a subcutaneous tissue of the user's body; j). ejecting of the needle back onto the wearable drug releasing pump; k). delivering of drug via the cannula of the wearable drug releasing pump.

### BRIEF DESCRIPTION OF THE DRAWINGS

Other objects, features, and advantages of the embodiment will be apparent from the following description when read with reference to the accompanying drawings. In the drawings, wherein like reference numerals denote corresponding parts throughout the several views:

FIG. 1, illustrates a schematic view of a wearable drug-releasing pump, according to an embodiment herein;

FIG. 2 illustrates an isometric view of an encoder unit (component of FIG. 1), according to an embodiment herein;

FIG. 3 illustrates an exploded perspective view of a syringe unit, according to an embodiment herein;

FIG. 4 illustrates an exploded view of an electronic unit, according to an embodiment herein;

FIG. 5A illustrates an exploded perspective view of a needle and a cannula unit, according to an embodiment herein; and

FIG. 5B illustrates an isometric view of a cannula insertion unit, according to an embodiment herein;

FIG. 6 illustrates a flowchart that depicts a method for providing of a drug into a user's body by the wearable drug-releasing pump, according to an embodiment herein.

### DETAILED DESCRIPTION OF THE DRAWINGS

Various embodiment of the present invention provides a wearable drug releasing pump for a user. The following description provides specific details of certain embodiments of the invention illustrated in the drawings to provide a thorough understanding of those embodiments. It should be recognized, however, that the present invention can be reflected in additional embodiments and the invention may be practiced without some of the details in the following description.

The various embodiments including the example embodiments are now described more fully with reference to the accompanying drawings, in which the various embodiments of the invention are shown. The invention may, however, be embodied in different forms and should not be construed as limited to the embodiments set forth herein. Rather, these embodiments are provided so that this disclosure is thorough and complete, and fully conveys the scope of the invention to those skilled in the art. In the drawings, the sizes of components may be exaggerated for clarity.

It is understood that when an element or layer is referred to as being “on,” “connected to,” or “coupled to” another element or layer, it can be directly on, connected to, or coupled to the other element or layer or intervening elements or layers that may be present. As used herein, the term “and/or” includes any and all combinations of one or more of the associated listed items.

Spatially relative terms, such as “top,” “bottom,” and the like, may be used herein for ease of description to describe one element or feature's relationship to another element(s) or feature(s) as illustrated in the figures. It is to be understood that the spatially relative terms are intended to encompass different orientations of the structure in use or operation in addition to the orientation depicted in the figures.

Embodiments described herein refer to plan views and/or cross-sectional views by way of ideal schematic views. Accordingly, the views may be modified depending on simplistic assembling or manufacturing technologies and/or tolerances. Therefore, example embodiments are not limited to those shown in the views but include modifications in configurations formed on basis of assembling process. Therefore, regions exemplified in the figures have schematic properties and shapes of regions shown in the figures exemplify specific shapes or regions of elements, and do not limit the various embodiments including the example embodiments.

The subject matter of example embodiments, as disclosed herein, is described with specificity to meet statutory requirements. However, the description itself is not intended to limit the scope of this patent. Rather, the inventors have contemplated that the claimed subject matter might also be embodied in other ways, to include different features or combinations of features similar to the ones described in this document, in conjunction with other technologies. Generally, the various embodiments including the example embodiments relate to a wearable drug releasing pump.

The term sway” or “sways” as used herein the context of the present disclosure refer to a motion of the encoder plate in left or right direction with respect to the lead screw.

As mentioned above, there remains a need for providing a wearable drug releasing pump that is small in size and capable to delivers insulin into a user's body. Accordingly, the present disclosure provides a wearable drug releasing pump that is capable to delivers the insulin.

The term first SMA wire (102A) and second SMA wire (102B) hereinafter collectively referred to and designated as “the one or more SMA wire (102)

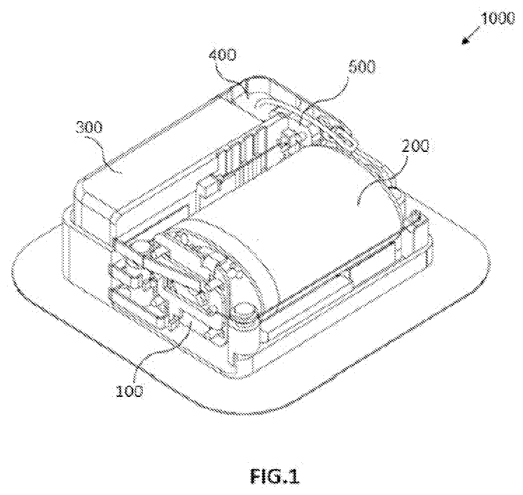

FIG. 1 illustrates a wearable drug-releasing pump (1000). The wearable drug-releasing pump (1000) includes a number of components and assemblies that work alongside for efficient functioning of the wearable drug-releasing pump (1000).

The wearable drug-releasing pump (1000) comprises an encoder unit (100), a syringe unit (200), an electronic unit (300), a needle and cannula unit (400), and a cannula insertion unit (500).

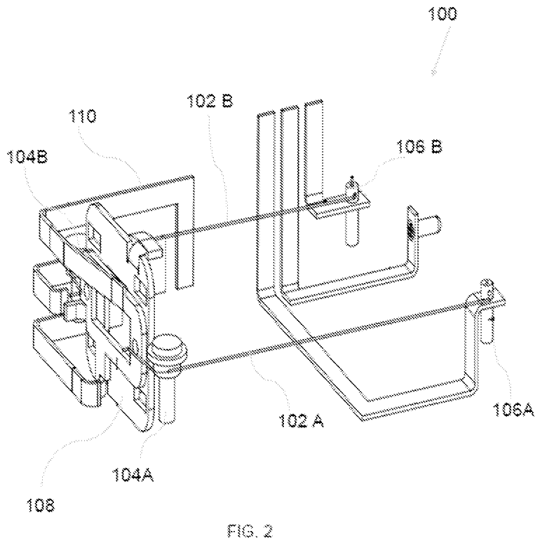

FIG. 2 illustrates the encoder unit (100) includes a one or more shape memory alloy wire (102) of which a first shape memory alloy wire (102A) (hereinafter individually referred to and designated as “the first SMA wire (102A)”, a second shape memory alloy wire (102B) (hereinafter individually referred to and designated as “the second SMA wire (102B), a one or more pulley (104) of which a first pulley (104A), a second pulley (104B) the one or more pulley (104) are placed adjacent to the encoder plate (108), a one or more locking stud (106) of which a first locking stud (106A), a second locking stud (106B), an encoder plate (108) and an encoder terminal (110), a microprocessor (not shown). The first SMA wire (102A) that are coupled to the first locking stud (106A) at one distal end and operatively coupled to the encoder plate (108) from another distal end by passing through the first pulley (104A). The second SMA wire (102B) that are coupled to the second locking stud (106B) at one distal end and operatively coupled to the encoder plate (108) from another distal end by passing through the second pulley (104B). The microprocessor (not shown) is operatively coupled to the encoder terminal (110).

In operation, the microprocessor (not shown) supplies current to the SMA wires (102) (according to the set current and set time). As a result, the SMA wires (102) heats up and is adapted to change in length, causing the encoder plate (108) to sway.

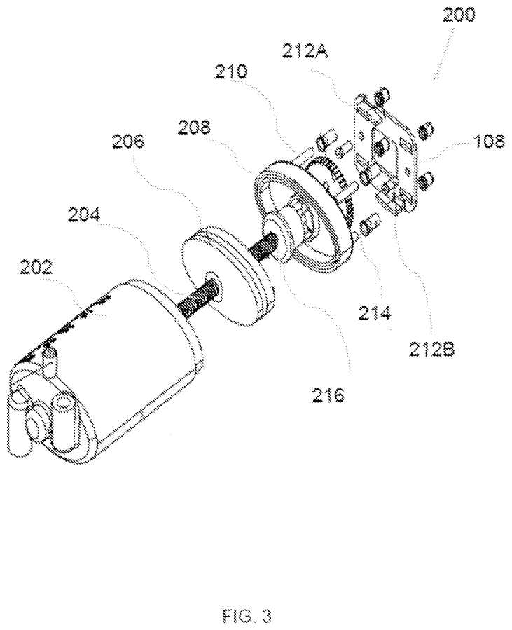

FIG. 3 illustrates a syringe unit (200) includes a syringe (202), a lead screw (204), a plunger (206), a back cap (208), a gear (210), a one or more pawl (212) of which a first pawl (212A), a second pawl (212B), a sleeve (214), a first end (216) and a fill port (not shown). The lead screw (204) having a first end (216) that is connected to the gear (210) and the second end (not shown) is on the opposite side of the first end (216) extend within the syringe (202). The plunger (206) is operatively coupled to the lead screw (204). The back cap (208) is coupled to the syringe (202). The gear (210) is operatively coupled to the encoder plate (108) by means of the first pawl (212A) and the second pawl (212B). The sleeve (214) is placed in between the back cap (208) and the encoder plate (108). The wearable drug releasing pump (1000) is having a fill port (not shown) by which the drug is fills by means of injection.

In operation, upon receiving the signal from the microprocessor (not shown) the first SMA wire (102A) heats up and contracts or vary in length, that is enable the movement of the encoder plate in left direction (108) and further the first pawl (212A) of the encoder plate (108) rotates the gear (210) in anti-clockwise direction. After few seconds the first SMA wire (102A) cool down and return to its original position. After that, upon receiving the signal from the microprocessor (not shown) the second SMA wire (102B) heats up and contracts or vary in length, that enables the movement of the encoder plate (108) in right direction and further the second pawl (212B) of the encoder plate (108) rotates the gear (210) in anti-clockwise direction.

Due to the rotation of the gear (210), the leadscrew (204) is rotating, and the plunger (206) is operatively coupled to the lead screw (204) that is configured to translate upon rotation of the lead screw (204) within the syringe (202) such that the plunger (206) upon translation dispenses a pre-determined quantity of a drug from the syringe (202), for example to dispense 0.3 ml of insulin from the syringe (202).

In some embodiments, the volume of the drug held within the syringe (202) may be within the range of 1.5 milli-meter (1.5 mm) to 2.5 mm.

In some embodiments, the predetermined quantity of the insulin dispensed by the syringe (202) lies within the range of 0.3 mm to 0.8 mm.

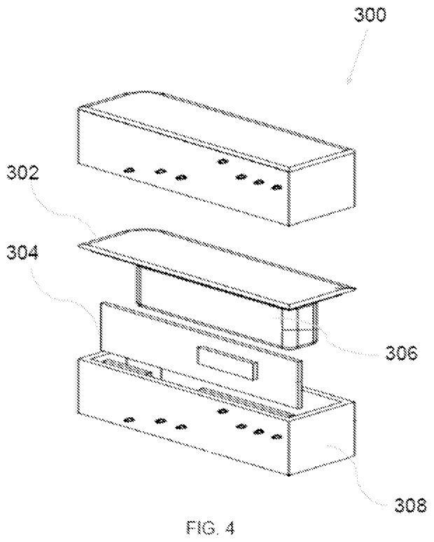

FIG. 4 illustrates an electronics unit (300) of the wearable drug-releasing pump (1000) houses the electronic components of the wearable drug-releasing pump (1000) which regulate the flow of the drug on the basis of predefined instructions. Further, the electronics unit (300) includes an electronics casting (308), electronic casting cap (302), a battery (304), a printed circuit board (PCB) (306)″. The battery (304) and the PCB (306) are placed in the electronic casing (308) and covered with the electronic casing cap (302). The electronic unit (300) is placed adjacent to the encoder plate (108) and configured to provide electricity to heat up the one or more SMA wires (102) upon heating the one or more SMA wires (102) is adapted to exhibit a variation in length such that upon variation of the length of one or more SMA wires (102) facilitates the encoder plate to sway.

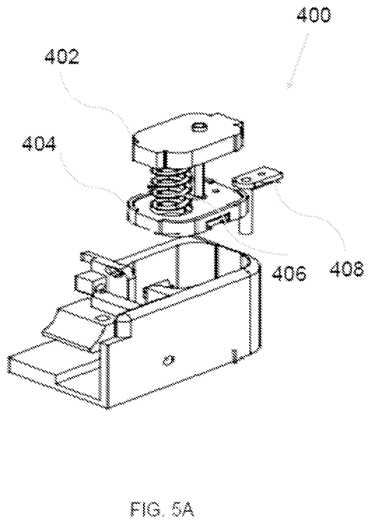

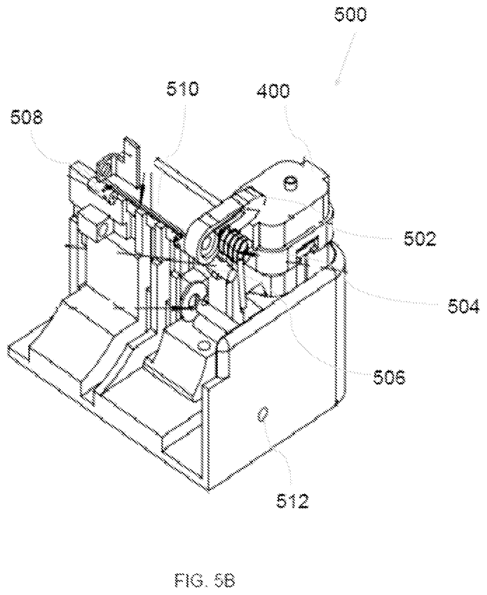

FIG. 5A illustrates a needle and cannula unit (400) that includes a needle holder (402) provided with a needle (not shown), a cannula holder (404) consisting of cannula (408), a spring (406) that is placed in between the needle holder (402) and cannula holder (404).

FIG. 5B illustrates the cannula insertion unit (500) that includes a needle insertor (502), a torsion spring (504), a third locking stud (506), a fourth locking stud (508), a push button (512) and a third SMA wire (510). The needle insertor (502) that is operably connected to the torsion spring (504). The third SMA wire (510) is connected to the third locking stud (506) on one end and to the fourth locking stud (508) on another end.

In operation, the user presses the button (512) by which it passes the signal to the microprocessor (not shown) that heats up the third SMA wire (510) by which the variation in length of third SMA wire (510) occurs that pulls the third locking stud (506) which further releases the torsion spring (504) that is attached to the needle inserter (502). Further, the torsion spring (504) pushes down the needle inserter (502) which pushes the needle (not shown) in needle holder (402), and the cannula (408) into the subcutaneous tissue of a user body. After the insertion the needle (not shown) is retracted back because of the spring (406), and the cannula holder (404) gets locked in the pod base slot (not shown) which holds cannula (408) inside the subcutaneous tissue of the user body.

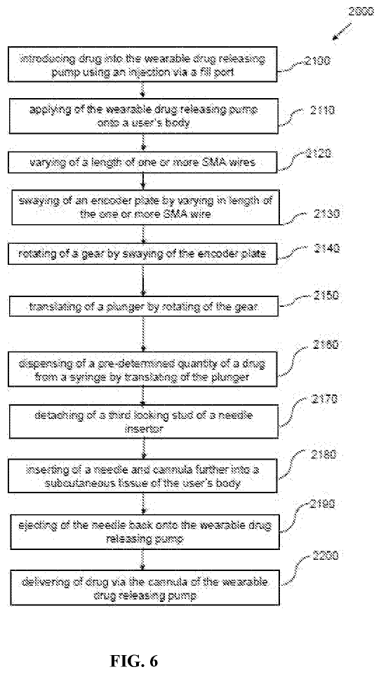

Referring to FIG. 6, which illustrates a flowchart describing a method (2000) for providing of a drug into a user's body using the wearable drug-releasing pump (1000), it may be observed that the system components of the wearable drug-releasing pump (1000) may be utilised in order to perform the method (2000) for providing of a drug into the user's body using the wearable drug-releasing pump (1000).

In which, the wearable drug-releasing pump (1000), may be introduced with the drug using an injection (not shown) via a fill port (not shown) at step 2100. The introduced drug may be stored in the syringe (202) of the syringe unit (200).

Once the drug is appropriately introduced onto the wearable drug-releasing pump (1000), the wearable drug-releasing pump (1000) may be applied to the user's body by removing an adhesive cover from the back of the wearable drug-releasing pump (1000) at step 2110 and sticking the wearable drug-releasing pump (1000) onto the user's body. Further, a variation in length of the one or more SMA wires (102) occurs upon receiving the electric current from the microprocessor (not shown) at step (2120), followed by swaying of an encoder plate (108) by varying in length of the one or more SMA wire (102) at step (2130), further followed by a rotating of the gear (210) by the swaying of the encoder plate (108) at step (2140) followed by translating of the plunger (206) by rotating of the gear (210) at step (2150) followed by dispensing of a pre-determined quantity of a drug from the syringe (202) by translating of the plunger (206) at step (2160) followed by detaching of the third locking stud (506) of the needle insertor (502) at step (2170), which in turn activates the insertion torsion spring (504), pushing down the needle inserter (502). The needle and cannula (408) may then be inserted into the subcutaneous tissue of the user's body by pressing the button (512) at step 2180, which in turn activates the insertion torsion spring (504), pushing down the needle inserter (502).

Due to a spring (406), the subcutaneous needle is ejected back into the wearable drug-releasing pump (1000) at step 2190, leaving the cannula (408) inserted inside the subcutaneous tissue of the user's body, via which the drug may be delivered to the user at step 2200.

In some embodiments, the microprocessor (not shown) of the encoder unit (200) of the wearable drug-releasing pump (1000) may store and relay the predefined drug requirements, also governing the amount of drugs to be delivered to the user as well as the rate at which the drug must be delivered to the user.

In some embodiments, the wearable drug-releasing pump (1000) may be connected to a software application installed onto a mobile device, wherein the user may input their daily drug requirements. Further, the user may also be provided with the option to make instantaneous changes to the daily drug requirements.

In some embodiments, the wearable drug-releasing pump (1000) may be small in size.

In some embodiments, the wearable drug-releasing pump (1000) may have a replaceable electronic unit (300).

In some embodiments, the wearable drug-releasing pump (1000) may have a replaceable cannula insertion unit (400).

In some embodiments, the wearable drug-releasing pump (1000) may worn on outside of the user's body.

In some embodiments, the mobile device may be a laptop, a computer, a mobile phone, a smart watch, or another smart device having input functionalities.

In some embodiments, the wearable drug-releasing pump (1000) may be connected to a software application installed onto a mobile device that tracks the daily food habits and insulin requirements of the user, and provide cognitive suggestions to input requirements respectively. Hence, the wearable drug-releasing pump (1000) may be configured to release variable dosages of the drug in case of emergencies and may relieve stress of the user to have multiple injections per day.

In some exemplary embodiments, the wearable drug-releasing pump (1000) delivers insulin uninterruptedly for improved glycaemic control.

In some embodiment, the wearable drug-releasing pump (1000) is configured to provide dosages for three or more days as per the patient's individual insulin needs.

The foregoing descriptions of exemplary embodiments of the present disclosure have been presented for purposes of illustration and description. They are not intended to be exhaustive or to limit the disclosure to the precise forms disclosed, and obviously many modifications and variations are possible in light of the above teaching. The exemplary embodiments were chosen and described in order to best explain the principles of the disclosure and its practical application, to thereby enable others skilled in the art to best utilize the disclosure and various embodiments with various modifications as are suited to the particular use contemplated. It is understood that various omissions, substitutions of equivalents are contemplated as circumstance may suggest or render expedient but is intended to cover the application or implementation without departing from the spirit or scope of the claims of the present disclosure.

1. A wearable drug releasing pump (1000) comprising:
an encoder plate (108);
one or more SMA wires (102) operatively coupled to the encoder plate (108); where the one or more SMA wires (102) are adapted to exhibit a variation in length in a manner that variation of the length of one or more SMA wires (102) facilitates the encoder plate to sway; and
a syringe unit (200) that comprises;
a syringe (202);
a gear (210) coupled to the encoder plate (108), and is adapted to rotate, upon sway of the encoder plate (108); a lead screw (204) having a first end (216) that is coupled to the gear (210), and configured to rotate, upon rotation of the gear (210); and
a plunger (206) coupled to the lead screw (204), and configured to translate upon rotation of the lead screw (204) within the syringe (202) such that the plunger (206), upon translation is adapted to dispense a pre-determined quantity of a drug from the syringe (202).

2. The wearable drug releasing pump (1000) of claim 1, comprises:
a plurality of studs (106A, 106B) that are configured to fix a one end of the one or more SMA wires (102); and
a plurality of pulleys (104) that are placed adjacent to the encoder plate (108), that are configured to provide a tension to the one or more SMA wires (102) such that another end of the one or more SMA wires (102) is fixed to the studs (106) projecting from the encoder plate (108).

3. The wearable drug releasing pump (1000) of claim 1, the encoder plate (108) comprises:
a one or more pawls (212A, 212B) projecting from the encoder plate (108) such that the one or more pawls (212A, 212B) are configured to rotate the gear (210), upon sway of the encoder plate (108).

4. The wearable drug releasing pump (1000) of claim 1, the syringe unit (200) comprises:
a lead screw (204) having a second end that is on a opposite side of the first end (216) of the lead screw (202) extends within the syringe (202).

5. The wearable drug releasing pump (1000) of claim 1, the syringe unit (200) comprises:
a fill port; that enables to fill the wearable drug releasing pump (1000) by means of injection.

6. The wearable drug releasing pump (1000) of claim 1, comprises:
an electronic unit (300) that is placed adjacent to the encoder plate (108);
the electronic unit (300) is configured to provide an electric current to heat the one or more SMA wires (102), upon heating the one or more SMA wires (102) is adapted to exhibit a variation in length such that upon variation of the length of one or more SMA wires (102) facilitates the encoder plate to sway.

7. The wearable drug releasing pump (1000) of claim 1, comprises:
a needle and canula unit (400) such that the needle and canula unit (400) comprises:
a needle holder (402) adapted to hold a needle;
a cannula holder (404) adapted to hold a cannula (408); and
a spring (406) that is disposed between the needle holder (402) and the canula holder (404) to retract back the needle.

8. The wearable drug releasing pump (1000) of claim 1, comprises:
a canula insertion unit (500) such that the cannula insertion unit (500) comprises;
a third locking stud (506); a fourth locking stud (508); a third SMA wire (510) that is coupled to the third locking stud (506) at one end and the fourth locking stud (508) on the other end;
a needle insertor (502);
a torsion spring (504) coupled to the needle insertor (502); wherein, variation of length of the third SMA wire (510) releases the third locking stud (506) that activates the torsion spring (504) by which the needle inserter (502) pushes down the needle holder (402) which further pushes down the needle and the cannula (408) into a subcutaneous tissue of a user body.

9. The wearable drug releasing pump (1000) of claim 1, is coupled to a Bluetooth Low Energy (BLE) enabled application to connect a user with the wearable drug releasing pump (1000).

10. A method (2000) for providing of a drug into a user's body by a wearable drug-releasing pump (1000), the method (2000) comprising:
a). introducing (2100) a drug into the wearable drug releasing pump (1000) using an injection via a fill port;
b). applying (2110) of the wearable drug releasing pump (1000) onto a user's body;
c). varying (2120) of a length of one or more SMA wires (102);
d). swaying (2130) of an encoder plate (108) by varying in length of the one or more SMA wire (102);
e). rotating (2140) of a gear (210) by swaying of the encoder plate (108);
f). translating (2150) of a plunger (206) by rotating of the gear (210);
g). dispensing (2160) of a pre-determined quantity of a drug from a syringe (202) by translating of the plunger (206);
h). detaching (2170) of a third locking stud (506) of a needle insertor (502);
i). inserting (2180) of a needle and cannula (408) further into a subcutaneous tissue of the user's body;
j). ejecting (2190) of the needle back onto the wearable drug releasing pump (1000);
k). delivering (2200) of drug via the cannula (408) of the wearable drug releasing pump (1000).
